(19) United States
(12) Patent Application Publication
(10) Pub. No.: US 2017/0246382 A1
(43) Pub. Date: Aug. 31, 2017

(54) METERING DEVICE FOR DISPENSING MEDICATION FLUID

(71) Applicant: MeaMedical AG, Riken (CH)

(72) Inventor: Hanspeter NIKLAUS, Riken (CH)

(73) Assignee: MeaMedical AG, Riken (CH)

(21) Appl. No.: 15/597,681

(22) Filed: May 17, 2017

Related U.S. Application Data
(63) Continuation of application No. PCT/EP2014/074907, filed on Nov. 18, 2014.

(51) Int. Cl. A61M 5/145; A61M 5/315

(52) U.S. Cl. CPC A61M 5/1456; A61M 5/14546; A61M 5/31593; A61M 2005/14573

(57) ABSTRACT

A metering device for dispensing medication fluid includes: a spindle unit, a drive unit for rotationally driving the spindle unit, and a reservoir for the medication fluid. The reservoir has a wall, which defines a cross-sectional area of the reservoir, and a plunger located in the reservoir. The rotational driving of the spindle unit causes a translational motion of the plunger, and therefore displaces medication fluid. Because the product of the cross-sectional area of the reservoir in the unit mm² and the spindle pitch in the unit mm/degree is less than 0.13 mm³/degree and the medication fluid is a liquid insulin having a concentration in a range of U20 to U100, a metering system can be formed that is suitable specifically for the CSII therapy of children and youth, where high discharge accuracy is important for good therapy control.

### TECHNICAL FIELD

The invention relates to a metering device for dispensing medication.

### STATE OF THE ART

Wearable injection devices and/or infusion devices are used for administering fluid medications, in particular insulin. The medication fluid is delivered continuously or quasi-continuously by means of a metering device containing a drive device for a plunger and having a reservoir with containing the fluid. The plunger in the reservoir is moved and the medication fluid in the reservoir is displaced and administered. Such devices are used as pump devices and manually operable “pens” in the treatment of insulin. For example, an “injection pen” is known from WO97/17095. It is true of both injection pens and insulin pumps that these devices must be as compact as possible, reliable and safe for the user and must be able to dispense small amounts of medication fluid in the most accurate possible manner and with the least possible error.

One example of an insulin pump is the D-TRON Plus pump from Roche Diabetes Care GmbH. It has a spindle unit fixedly arranged in the pump, formed by three telescoping spindle stages. The first displacement stage, which is movable against the plunger of the reservoir, can only execute a forward motion. A second displacement stage can execute a forward motion as well as a rotational motion when entrained by a drive stage. The drive stage only executes a rotational motion to create the forward motion of the first or second displacement stages. The drive device with its fixedly connected spindle unit of the D-TRON Plus pump is described in WO98/47552. Since the introduction of insulin pumps, the design, specifically that of the metering devices, has hardly changed noticeably at all. The first generation of traditional insulin pumps has a design for the metering device, such as that known from the D-TRON Plus pump from Roche Diabetes Care GmbH. A motor drives a spindle unit either directly or indirectly by way of a gear reduction ratio, wherein the spindle unit has at least one spindle drive. The spindle unit consists of a spindle nut and spindle rod, wherein either the spindle nut is driven so that the spindle rod can be extracted and exert an impact force on a plunger of the reservoir to move it forward, or alternatively, the spindle rod may be driven to rotate and then the spindle nut is driven axially to advance the plunger in this way. Pumps of the first generation have reservoir volumes between approximately 1600 mm³ and 3200 mm³ and thus have a capacity of approximately 160 to 320 IU U100 insulin. The spindle pitches vary between about 1.0 and 1.2 mm/revolution and the cross-sectional areas for displacement of medication fluid amount to between approximately 65 and 115 mm². With this generation of metering pumps, the spindle unit is arranged in the reusable part of the pump. This has the consequence that the spindle unit is always arranged in the pump housing and must always be brought back into the starting position by a reverse movement, after which the metering device can again be loaded with a freshly filled ampoule. In order for the time for retraction of the spindle unit to be within a period of time that is acceptable for the user, the spindle pitches cannot be designed to be as small as desired in general. In practice, spindle pitches of 1.0 to 1.2 mm/revolution have proven successful for such metering devices. The required time for retracting the spindle is indirectly proportional to the spindle pitch. In other words, the smaller the spindle pitch, the more time is required for retraction of the spindle unit into this starting state. The following table shows the cross-sectional area for displacement of medication fluid and the spindle pitch for traditional metering devices.

Metering Devices of the First Generation:

TABLE 1

| | Cross- | | |
|---|---|---|---|
| | sectional | Equivalent | |
| | area | diameter | Spindle pitch |
| | of reservoir | of reservoir | in |
| | in mm² | in mm | mm/revolution |
| Roche D-TRON | 67.20 | 9.25 | 1.2 |
| Plus | | | |
| Roche Spirit/ | 113.10 | 12.0 | 1.2 |
| SpiritPlus | | | |
| Medtronic | 113.10 | 12.0 | 1.0 |
| Minimed | | | |
| Paradigm, Veo | | | |
| Animas Vibe, | 95.03 | 11.0 | 1.0 |
| 2020 | | | |
| Deltec Cozmo | 113.10 | 12.0 | 1.0 |

The first generation of metering devices in the form of insulin pumps has in common the fact that the fluidic path leads from the ampoule into the subcutaneous tissue of the user by way of a catheter and a cannula. For example, the user may carry the pump in his pants pocket, so that medication fluid is displaced by the forward motion of the plunger and dispensed to the user via the catheter.

The most recent generation of metering devices is worn directly on the user's body. A catheter for the fluidic connection of pump and user is no longer necessary for such devices. One exemplary embodiment of the latest generation—hereinafter referred to as the second generation—is the patch pump Omnipod from the company Insulet Corporation. According to the manufacturer's information, the reservoir volume for this patch pump, which is available commercially, is 2000 mm³ for holding 200 IU of U100 insulin. Another second-generation patch pump which is under development is the “MeaPump” by the same applicant, known from PCT/EP2014/059889. This pump likewise has a reservoir volume of 2000 mm³ for holding 200 IU of U100 insulin, wherein this pump is designed with a telescoping spindle integrated into the plunger. In the advance of the telescoping spindles, both spindle drives move outward at the same time, so that the spindle pitch can be calculated by adding the pitches of the two spindle drives. Each spindle drive has a pitch of 0.5 mm/revolution, so that the total pitch amounts to 1.0 mm/revolution. The metering device from Insulet Corp. is described in WO2013/149186, which relates to a spindle rod fixedly connected to the plunger and a spindle nut around the spindle rod, wherein the plunger is in its end position as the starting position, i.e., before being filled with insulin. During filling, the plunger together with the spindle rod is moved in reverse, wherein a coupling releases the connection between the spindle rod and the spindle nut for the filling operation. In pump delivery, there is a coupling between the spindle nut driven by a motor unit and the rotationally secured spindle rod. The spindle rod executes a forward movement with the rotation of the spindle nut, so that the plunger connected to the spindle rod is displaced and thereby dispenses medication fluid to the user.

### Metering Devices of the Second Generation:

TABLE 2

| | Cross- | | |
|---|---|---|---|
| | sectional | Equivalent | |
| | area of | diameter | Spindle pitch |
| | reservoir in | of the reservoir | in |
| | mm² | in mm | mm/revolution |
| Insulet Corp., | 125 | 12.62 | 0.4 |
| Omnipod | | | |
| MeaMedical AG, | 125 | 12.62 | 1.0 |
| MeaPump | | | |

The Solo M pump from Roche Diabetes Care GmbH is another patch pump currently in development. This pump may also have a reservoir with a capacity of 2000 mm³, i.e., 200 IU of U100 insulin. The patch pumps of the second generation in general have smaller spindle pitches than those of the first generation. Conversely, the cross-sectional areas of the reservoirs have larger dimensions than the cross-sectional areas of the reservoirs of the first generation. The dimensions and the shape of the cross-sectional areas for the second generation have been adapted to the requirements of patch pumps, wherein the latter should be as shallow as possible and with a compact length. Due to these specifications, reservoir areas have varied from cylindrical areas to elliptical areas, and moreover, the cross-sectional area of the second generation has increased in general in comparison with the first generation due to the reduced length of the device.

The first-generation metering devices have the disadvantage that very small amounts of medication fluid cannot be dispensed with sufficient accuracy. This is due to the fact that the smallest basal rate increment, which amounts to 0.0025 IU of U100 insulin (0.025 mm³) with the SpiritPlus pump, for example, corresponds to exactly one motor step. The smallest basal rate with the SpiritPlus pump is 0.05 IU/h at a concentration of U100, wherein there are 20 pump intervals of 3 minutes each. This yields a smallest basal rate increment of 0.0025 IU, which is pumped within 3 minutes. The motor for the SpiritPlus pump is a motor that is controlled by Hall sensors and has an increment size or resolution of 60 angular degrees. With short triggering, it may happen that, due to the low inertia of the drive unit, the metering system is unable to move by just one motor step. In concrete terms, this means that the motor may execute four motor steps, for example, for the desired movement of one step. As a result, no more medication fluid is conveyed for metering intervals after 6 minutes, 9 minutes and 12 minutes. The metering device is triggered by a control unit to execute another motor step only after a period of 15 minutes, and here again, an inaccuracy of several motor steps is to be expected. Such systems are not optimal in particular for users requiring only very small amounts of medication fluid. On the average, the error in metering the medication is corrected with an increase in treatment time, but extremely small basal rates or small bolus doses of medication fluid cannot be dispensed with sufficient accuracy due to the inaccurate positioning of the motor.

It is fundamentally true that all metering devices of the first generation have the problem described here. In other words, extremely small metering amounts can be set by the user but there are major dispensing errors because the angular resolution of the driving motor is of the same order of magnitude as the metering increment itself. In other words, the smallest metering increments correspond to the resolution of the system so in practice for such extremely small metering increments, the metering errors may be too high. The metering devices of the second generation in the form of patch pumps have not been improved with regard to this problem. These are merely systems having a smaller spindle pitch. Such metering devices of the second generation therefore also do not meet the requirements for metering accuracy with extremely small metering rates and metering increments, as is desirable for treatment of children and young people in particular. These users in particular require systems that can administer medication fluid with precision for extremely low basal rates, basal rate increments and small bolus doses of medication fluid. This problem of how to administer extremely small metering units accurately is well known, in particular in the treatment of children and young people in insulin pump therapy. The only method used in practice to correct this problem is to dilute the insulin concentration. The insulin concentration in general is U100 insulin, so that a volume of one milliliter contains 100 IU insulin (International Units). For comparison, a dilute insulin with a U40 insulin concentration contains 40 IU per milliliter of medication fluid. In a complicated procedure, U40 insulin is prepared manually because practically no dilute insulin preparations are available from manufacturers. To prepare U40 insulin, for example, 4 milliliters of U100 insulin and 6 milliliters of diluting medium are injected into an empty vial. It must pointed out here that it is important to use the correct diluent for the corresponding U100 insulin. Likewise, after dilution the container with the diluted insulin must be labeled immediately so that there is no confusion later when using the diluted medication fluid. It is obvious that dilution of insulin is a tedious procedure which is subject to handling errors, so that there may be substantial risks in treatment in the event of careless procedures. When changing from U100 insulin to U40 insulin, the metering device must ultimately be reprogrammed, so that it will dispense the correct amounts of insulin in accordance with the insulin concentration used. Dilution of U100 insulin is thus complicated and is not protected from handling errors on the part of the user. Dilution of insulin is the only method available today for improving the metering accuracy. Diluting the concentration results in an improvement in the dispensing accuracy but dilution is complicated, is not suitable for all insulin, and is subject to errors in use, so that it may result in serious risks for the user.

The first generation devices referenced in the introduction are described in WO98/47552, WO00/25844, WO03/074110 and WO02/02164. These devices have in common the fact that the spindle unit for driving the plunger is arranged in a fixed and reusable housing. It was described in detail in the introduction that these devices are not optimal with regard to accuracy in dispensing extremely small basal increments and extremely small bolus doses because these small amounts of medication fluid correspond to the resolution of the metering devices themselves. Since position errors with the driving motors are of the same order of magnitude as the required positioning of these motors for dispensing extremely small metering increments, large dispensing errors may occur here. These devices are therefore not optimal for care of children and young people with insulin in continuous subcutaneous insulin infusion—hereinafter referred to as CSII therapy. In many cases, the recommendation is to dilute the insulin to thereby achieve a better accuracy for dispensing of extremely small metering amounts. However, dilution of insulin must be considered critically in this regard because it must be performed manually and considerable handling errors by users may occur in the dilution process.

Second generation devices are known from WO2013/149186 and WO2011/007356. The device described in WO2013/149186 is used only once which means that after being used for a maximum of 3 days, the user must dispose of the entire device together with the batteries. The second generation devices have been improved only to the extent that the spindle pitches can be reduced in comparison with those of the first generation. The second generation devices have spindle pitches of 0.4 mm/revolution to 1.0 mm/revolution while the first generation devices have pitches of 1.0 mm/revolution to 1.2 mm/revolution. Since the second generation devices have large cross-sectional areas for the reservoirs to thereby achieve the most compact possible designs, the advantages of the smaller spindle pitches are leveled out again so that even these devices belonging to the second generation are still not optimal for CSII therapy in children and young people. The second generation devices have cross-sectional areas of approximately 125 mm² while the devices of the first generation have cross-sectional areas of approximately 65 mm² to 115 m².

The metering devices of the first and second generations mentioned above are metering devices with one ampoule as a reservoir, wherein medication fluid is dispensed to the user by displacement of the plunger in the reservoir.

Approaches in which a separate metering cylinder is provided downstream from the reservoir represent a new approach for accurately administering medication fluid. These metering devices are referred to below as systems of the third generation. One example of this type is described in WO2012/140063, where instead of displacing the plunger in a reservoir, medication fluid is conveyed from a reservoir to a metering device by suction by way of a unit and then is dispensed to the user via the metering device. WO2012/140063 discloses that the volume of the metering cylinder may correspond to 4% ( 1/25) of the reservoir. For reservoirs with 200 to 300 IU insulin, which are preferably used in insulin pumps, this means that the metering cylinder may have a capacity of 8 to 12 IU insulin. This specification additionally discloses that the metering cylinder may have a capacity amounting to 20% of the total daily dosage TDD. The average user requires 50 IU insulin per day. According to this the amount of insulin in the metering cylinder should be 10 IU insulin. It is a disadvantage to be constantly switching from a fluidic connection to the reservoir for filling up the metering cylinder and to dispensing insulin to the user. This is technically complicated to accomplish. Due to the metering cylinder being raised constantly by reversing the direction of the plunger and then subsequently dispensing the fluid to a user, the metering device must execute an increased total stroke for distributing a reservoir for dosed dispensing of the reservoir. This larger stroke distance for distributing a reservoir has a negative effect on the energy balance of the metering device. Measures must also be taken, so that a position error due to thread play occurring when switching from pulling up the plunger to ejecting the medication fluid and thread play in the drive train can both be compensated. Constant switching in pulling up the plunger and subsequently discharging insulin may result in a hysteresis in the delivery. Hystereses in the discharge result in reduced delivery, so that the user is not optimally supplied with medication fluid and the therapy result turns out to be inadequate. Likewise, it must also be pointed out here that the small dimensions of the diameter of the metering cylinder may have negative effects on the precision in dispensing. Thus, a metering cylinder with a diameter of 5.0 mm and a diameter tolerance of ±0.05 mm has an area error of ±2%. However, one ampoule of the first generation with a diameter of 10.0 mm and the same diameter tolerance of ±0.05 mm will have an area error of only ±1%. The area error is essential for medium and larger metering amounts and, for example, results in a substantial dispensing error over a 24-hour period, during which the metering cylinders having smaller dimensions will even have a greater dispensing error than the reservoir plunger of a first-generation metering device. It is pointed out clearly here that the third-generation systems constitute a new design. With the systems of the first and second generations as well as with the present invention, the reservoir serves both as a storage container for the medication fluid and as part of the metering device, because the plunger of the reservoir is itself displaced. There is a strict separation in the third-generation systems. The reservoir serves only as the storage container for the medication fluid, while the metering cylinder is only responsible for dispensing the medication fluid downstream from the reservoir. EP 2361646B1 describes another exemplary embodiment of a metering device of the third generation. It is stated in that patent that the metering cylinder has a capacity for a metering amount of 4 to 20 IU, which corresponds to 40 to 200 mm³ when using U100 insulin. It is also stated that ratio of length to diameter of the metering cylinder may be 10:1 to 1:1. A range for the metering diameter from 3.70 mm to 6.34 mm can be calculated from this. The metering system may also have a spindle pitch of 0.5 mm/revolution to 2.0 mm/revolution. Furthermore, it is stated that the reservoir may have a capacity of 200 to 500 IU of U100 insulin, which corresponds to a volume of 2000 to 5000 mm³ for a U100 insulin. EP 2361646B1 also describes a metering device for the average user, who may need 50 to 100 IU insulin per day. Consequently, the reservoir would have large dimensions. EP 2361646B1 makes no statements about how metering areas and pitches can be optimized and/or combined to allow formation of the most accurate possible metering device for CSII therapy of children and young people. As a representative of the third generation, EP 2361646B1 is based on the assumption that the reservoir and the metering cylinder must be separated from one another in order to be able to form the most accurate possible metering device. The present invention is directed at a novel path, which is contrary to the prior art and takes as its object the goal of improving a metering device of the first or second generation in such a way that it is optimally suitable for CSII therapy of children and young people with respect to metering accuracy and the size of the reservoir volume, i.e., the dose amount.

Metering devices such as “injection pens” or “pens” are known from WO97/17095, where the metering device consists of an ampoule with a metering area F and a manually operable spindle unit. The spindle unit in WO97/17095 makes it possible to set metering increments. With regard to the resolution of the metering increments, it should be pointed out that the metering knob has metering increments in the form of quarter rotations of the metering knob. In a first step, the user rotates the knob by the desired number of quarter rotations and then pushes the metering knob, so that the medication fluid is dispensed. It is known that injection pins have a limited resolution. Commercial devices have metering increments of 0.1 IU insulin. Consequently, injection pens for children and young people suffering from type 1 diabetes can be used only to a limited extent. Insulin pumps are more suitable for this group of users, because these pumps can deliver basal rates of 0.05 to 0.1 IU/h more or less continuously.

WO03/017914 describes a glass ampoule having optimized diameter tolerances, so that 20 insulin doses of 1 IU each can be administered with the tolerance required by the ISO standard. The insulin concentration used here is U200, so that higher requirements are made of the tolerance of the ampoule diameter. The analysis presented in WO03/917914 shows that, for an insulin with a concentration of U200, the diameter must be between 7.45 mm and 9.25 mm in order to meet the requirement of the standard that specifies that the total dose of 20 IU dispensed must be within a tolerance of ±1 IU after 20 metering increments of 1 IU. Such an ampoule designed for dispensing U200 insulin is not suitable for use in CSII therapy in children and young people, where the daily doses of required insulin—the daily insulin demand or the total daily dosage TDD—are in the range of 5 IU to 20 IU. In general, the total daily dosage must be estimated on the basis of body weight. The body weight in kg here is multiplied times a factor of 0.5 IU/kg·day. For a small child weighing 10 kg, this yields a total daily dosage of 5.0 IU, where approximately 50% is provided for the basal administration and 50% for the bolus dose for compensation of carbohydrate units with meals. Ultimately, an average basal rate of 0.1 IU/h can be calculated (2.5 IU/24 h), wherein the basal rate may be substantially below the average basal rate at night. The ampoule with an optimized diameter of 1 IU U200 insulin for accurate dispensing according to WO03/017914 is therefore not suitable for small children and young people. WO03/017914 described that the state of the art for U100 insulin is 1.5 mL prefilled glass ampoules with a diameter of 6.85 mm and 3.0 mL prefilled glass ampoules with a diameter of 9.25 mm. Such glass ampoules are used primarily in injection pens whose mechanical metering systems have a mechanical displacement accuracy of only ±0.083 mm for the plunger in dispensing 1 IU. According to WO03/017914, improved systems have an accuracy of ±0.055 mm. For a mechanical injection pen with a displacement accuracy of ±0.055 mm in combination with a glass ampoule having a diameter of 6.85 mm, a dispensing error of ±0.202 IU can be calculated as a result of the displacement tolerance for an insulin concentration of U100. Such metering devices are therefore not particularly suitable for children and young people. Insulin pumps that can be loaded with prefilled glass ampoules of the type described here are known in the prior art. One example is the D-TRON Plus pump, which uses a prefilled glass ampoule with an inside diameter of 9.25 mm. Only prefilled glass ampoules with a diameter of 9.25 mm are used in insulin pumps. In order for the longitudinal extent of the D-TRON Plus pump to remain compact, this pump has a telescoping spindle. The corresponding metering device belong to said first generation of insulin pumps in which the spindle is arranged in a fixed housing and the spindle pitch has values between 1.0 mm/revolution and 1.2 mm/revolution to limit the return time. Prefilled glass ampoules with a diameter of 6.85 mm and a volume of 1500 mm³ have been used only in manually operated injection pens and have the dispensing tolerance of ±0.202 IU, which was derived previously. The analysis described in WO03/017914 takes into account only the diameter tolerances and the displacement tolerances, i.e., the stroke tolerances, as a result of spindle pitch errors, for example, for the plunger of the mechanical metering systems. The displacement tolerances of mechanical injection pens are mentioned here. WO03/017914 does not make any statements about the metering accuracy of insulin pumps, but merely postulates that the metering systems of insulin pumps having a higher accuracy must always conform to the aforementioned standard because the less accurate mechanical metering systems of injection pins already conform to the ISO 11608-1 Standard. Thus, WO03/017914 does not contain any statements about the accuracy of metering systems in insulin pumps, nor are there any statements about how such a system can be improved with regard to accuracy. Therefore, the prior art according to WO03/017914 is not suitable for a metering system in the CSII treatment of children and young people. The glass ampoule filled with U200 insulin, as disclosed in WO03/017914, is therefore also not suitable for CSII therapy in children and young people.

In addition, WO2008/055689 should also be mentioned here for the sake of thoroughness. This patent specification discloses a method, wherein the diameter of the ampoule is adjusted as a function of the insulin concentration. The basis is an ampoule with a diameter of 9.65 mm for administration of U100 insulin. The area is reduced by one-half if the concentration is doubled, and in the opposite case, the area is doubled if the concentration is reduced by one-half. This achieves the result that, with the same displacement of the plunger, the same amount of insulin can be administered. The invention according to WO2008/055689 does not solve the problem of the metering accuracy of extremely small doses because the result is always the same stroke for all combinations of area and concentration. A positioning error of the drive unit thus still have the same effect for all the proposed pairings of area and concentration, so that no improvement can be achieved with regard to the positioning error of the motor.

The metering devices of the first generation have volumes of approximately 1600 mm³ to 3500 mm³ and the metering devices of the second generation have volumes of only approximately 2000 mm³. Prefilled glass ampoules for injection pens have volumes of approximately 1500 mm³ to 3500 mm³. The size of the reservoir volumes and the parameters of the state-of-the-art metering devices were designed by taking into account the needs of average users. The average user has a daily insulin dose of 50 IU/day to 100 IU/day. In order for an average user to be able to use a metering device without changing the reservoir for two to four days, reservoirs volumes of approximately 200 to 300 IU, i.e., approximately 2000 mm³ to 3000 mm³ are needed. Neither the reservoir volumes nor the mechanical drive systems of the metering devices have been adapted specifically to the therapeutic needs of children and young people.

### DESCRIPTION OF THE INVENTION

The purpose of the invention is to improve upon a metering device for accurately dispensing medication fluid with regard to the dispensing accuracy of extremely small basal rate increments and small bolus doses, such as those desired in CSII therapy for children and young people in particular. Due to the accurate dispensing of small metering amounts, the therapeutic result, for example, control of the blood sugar level, can be improved. The goal is also to optimize the metering device, so that even insulins with a maximum concentration of U100 can be used in CSII therapy of children and young people without requiring dilution of the medication fluid. The purpose of the present invention is to develop systems of the first and second generation with regard to metering accuracy so that they are suitable in particular for CSII therapy of children and young people.

In an embodiment, the metering device of the invention has a spindle unit with a constant spindle pitch, a drive unit for rotational drive of the spindle unit and the reservoir for the medication fluid. The reservoir also has a wall, which defines a cross-sectional area of the reservoir and has a plunger in the reservoir, wherein the rotational drive of the spindle unit causes a translational movement of the plunger, so that the plunger can be moved relative to the wall of the reservoir to displace the medication fluid. A metering device which is especially suitable for CSII therapy of children and young people can be created due to the fact that the product of the cross-sectional area of the reservoir, expressed in units of mm², and the spindle pitch, expressed in units of mm/angular degree, amounts to less than 0.13 mm³/angular degree and the medication fluid is a liquid insulin in a concentration of U20 to U100. On the one hand, the sensitivity of the metering device with respect to angle errors of a drive unit can be minimized. The sensitivity of a parameter is a measure of how much its tolerances can affect an output variable. In the present invention, the dose of medication fluid dispensed corresponds to the output variable or the target variable to be controlled. The independent variables may be the motor angle, the spindle pitch and the equivalent reservoir diameter. For each independent parameter, the corresponding sensitivity can be determined, as described in detail in the following description of the invention. Theoretically, the invention is based on the finding that the sensitivity for the angle error of the drive unit is proportional to the product of the cross-sectional area of the reservoir and the pitch of the spindle unit and is proportional to the insulin concentration. These relationships make use of the present invention to create a metering device having a minimized error in dispensing extremely small dose amounts. This is achieved by the fact that, in comparison with the prior art, the product of the cross-sectional area and the spindle pitch is optimized, so that, up to insulin concentrations of max. U100, the metering device according to the invention can be suitable for CSII therapy in children and young people. The metering device according to the invention is suitable in particular for CSII therapy in children and young people with type 1 diabetes. The therapeutic result can be improved due to the improved accuracy in dispensing extremely small basal rate increments and small bolus doses. The metering device according to the invention therefore makes it possible to accurately administer small and extremely small metering amounts in CSII therapy for children for juveniles by reducing the angle error due to a reduction in the corresponding sensitivity. The invention has theoretically discovered a therapeutic basis and recognized that the sensitivity for the angle error is proportional to the product of the cross-sectional area and the pitch. Said sensitivity is also proportional to the insulin concentration. Therefore, the invention proposes optimizing the product of the cross-sectional area and the spindle pitch in comparison with the prior art, i.e., minimizing this product, so that when using insulins with concentrations of U20 to max. U100, CSII therapy for children and young people can be greatly improved and therefore a better therapeutic result, i.e., better blood sugar control, can be achieved.

Advantageous embodiments of the invention are also disclosed.

In the treatment of diabetes, a liquid insulin in a concentration of U100 is preferably used because most manufacturers offer only U100 insulin. In addition, the reservoir may have a capacity for 20 to 100 IU medication fluid, so that the volume is in a range of 200 to 1000 mm³ for an insulin concentration of U100. This amount of medication is sufficient to cover the insulin demand for 2 to 4 days without having to refill the reservoir or replace it. This preferred metering device therefore makes it possible that the insulin in the reservoir is sufficient for use for 2 to 4 days in CSII treatment of children and young people; that the insulin used in an initial concentration of U100 need not be diluted to improve the dispensing accuracy and therefore U100 insulin can be used directly, and small and extremely small metering amounts can be administered with precision due to the fact that the angle error is reduced by reducing the corresponding sensitivity. First, this makes it possible to omit the step of diluting the insulin, so that handling of the metering device is greatly simplified. Second, due to the improved dispensing accuracy with extremely small basal rate increments and small bolus doses, the therapeutic result can be improved. A metering device with which U100 insulin can be used may also have a more compact design than metering devices using lower insulin concentrations.

The maximum sensitivity for the diameter error in the cross-sectional area can be defined by a lower limit for the diameter, so that the metering device according to the invention can dispense large metering amounts with a defined maximum error. The cross-sectional area preferably has an area amounting to more than 24 mm². The sensitivity for the diameter error is proportional to the inverse of the diameter.

The metering device may preferably have a cross-sectional area in the range of 24 mm² to 58 mm², wherein the equivalent diameter is in a range from 5.5 mm to 8.5 mm. If the diameter is reduced too much, the average dispensing error over a long interval of time of 24 hours, for example, is increased. It has been found that the preferred range for the equivalent diameter from 5.5 mm to 8.5 mm in particular has optimum properties with regard to tolerances for the accuracy of the long-term dispensing but also with regard to the producibility of an actual metering device having such dimensions. The spindle pitch of the metering device may preferably be in the range from 0.2 mm/revolution to 1.0 mm/revolution, i.e., in a range from 0.00056 mm/angular degree to 0.0028 mm/angular degree after converting from mm/revolution to mm/angular degree. The product of the preferred cross-sectional areas and the preferred spindle pitches yields a preferred design range for the metering device according to the invention. In particular with these preferred combinations of cross-sectional area and spindle pitch, the result is a minimized sensitivity with respect to the angle error of the drive unit, so that systems designed in this way are especially suitable for CSII therapy of children and young people. Another favorable design range is 6.4 to 7.5 mm for the equivalent diameter and 0.3 to 0.9 mm/revolution for the spindle pitch.

A design range for the metering device in which the product of the cross-sectional area of the reservoir in units of mm² and the spindle pitch of the spindle unit in units of mm/angular degree is less than 0.08 mm³/angular degree, and the cross-sectional area is greater than 32.2 mm², which corresponds to a diameter of 6.4 mm, is especially suitable for insulin pumps and their use in CSII therapy in children and young people. Therefore, a system that also has a reduced sensitivity for the diameter error can be created, so that large metering amounts can be dispensed with greater precision. Furthermore, the preferred metering system has a reduced sensitivity to angle errors of the motor so that small and extremely small metering amounts can be dispensed with a high precision. Therefore a further improvement in dispensing extremely small metering increments can be achieved in comparison with the prior art. In addition, the long-term error in dispensing can be further improved in comparison with systems of the third generation.

In a preferred specific embodiment, the drive unit has a motor as a drive element and has a motor-driven gear with a gear reduction ratio for step-down of the motor angle. Gear reduction ratios in a range from 200 to 2000 are especially favorable for the motor angle. The sensitivity with respect to a motor angle error can be further reduced by means of such high gear reduction ratios. It is advantageous if the spindle unit is formed from only one spindle nut and one spindle rod. It is then possible for the spindle nut to be driven rotationally and for the spindle rod to be extracted, or for the spindle rod to be driven rotationally, which leads to extraction of the spindle nut.

In a particularly advantageous embodiment, the spindle unit is arranged in the plunger itself and is driven by a driving rod arranged on the gear output, so that the driving rod has an axial stop for support of the spindle unit in the delivery state. Due to the fact that the spindle unit is integrated into the plunger, the spindle unit may be used for just a single application with the reservoir. Furthermore, it is advantageous for the spindle unit to be supported on the driving rod, because in this way, a force acting on the spindle unit can be determined by means of a force sensor arranged beneath the driving rod. This force can then be used by a control unit for monitoring the reservoir with respect to occlusions. Due to the integration of the spindle unit into the plunger, the metering device may also hold partially filled reservoirs. By reverse operation, the spindle unit can be supported on its stop, which is formed on the driving rod and can subsequently dispense medication fluid in dosed form. Since the force sensor can detect contact with the driving rod, a control unit can determine the amount of medication fluid in the reservoir. The amount of medication fluid can therefore be calculated, because the total lift of the spindle unit is the same for all reservoirs and corresponds to an equivalent number of motor steps. After the spindle unit has come in contact with the stop provided on the driving rod, the number of motor steps carried out until contacting the stop can be subtracted from the total of motor steps. The remaining motor steps correspond to the motor steps still available for dispensing medication fluid. The remaining amount of medication in the reservoir can be determined from these motor steps at any point in time.

It is particularly advantageous if the spindle rod is fixedly connected to the plunger and the spindle rod is driven by the driving rod. An external thread on the spindle rod engages with an internal thread on a twist-secured spindle nut and thus forms a spindle drive.

Two scenarios may be considered for filling this preferred specific embodiment. The spindle rod can preferably be retracted in a starting state before filling the reservoir, and the plunger together with the spindle unit may be displaceable for filling the reservoir with a pull-up rod. In this variant, the user fills the reservoir manually, connecting the reservoir to a storage container by means of an adapter, so that the reservoir and the storage container are in fluidic communication. In a first handling step, the air in the reservoir is displaced into the storage container. Then the user can move the plunger in reverse by means of the pull-up rod, so that medication fluid flows from the storage container into the reservoir and thereby fills the reservoir. Filling of the reservoir, which can take place largely automatically by means of the metering device, is preferred in particular. In this advantageous specific embodiment, the spindle rod is initially in an extracted position and thus the plunger is in its topmost position. The user first connects the reservoir to the drive unit by inserting the reservoir into a housing and bringing the spindle rod of the reservoir into engagement with the driving rod of the drive unit. Next, the user establishes the fluidic communication between the reservoir and the storage container by first connecting the adapter, which has a connecting needle, to the reservoir and then connecting the storage container to the adapter, so that fluidic communication is established by way of the connecting needle. By reverse rotation of the driving rod, the plunger can be moved in reverse, so that medication fluid overflows into the reservoir. In reverse operation, a control unit can determine the motor steps and he automatically determine the amount of medication fluid in the reservoir on the basis of this information. Axial support of the spindle nut on the wall of the reservoir is especially advantageous. Support on the wall during filling has the advantage that no axial force can act on the drive unit during filling.

Filling with a pull-up rod is especially advantageous because then the plunger with its integrated spindle unit is displaced as a whole. During the filling, no relative displacement occurs between the plunger and the spindle unit and the elements of the spindle drive, such as the spindle nuts and spindle rods. Due to such a particularly advantageous arrangement of the spindle unit in the plunger, it is possible to calculate the remaining number of motor steps on the basis of the constant total stroke and the number of motor steps until the spindle unit runs up against its stop. Ultimately, the remaining amount of medication fluid still available can therefore always be determined at any point in time. This constitutes a great simplification in handling because in this way the user need no longer enter into a control unit the value of the initial amount of medication fluid when the reservoir is partially filled. For both filling methods—filling by hand via pull-up rod or automatic filling via drive unit—the amount of medication fluid in the reservoir can thus be determined automatically by a control unit after filling. This is a significant improvement for the user and leads to greater safety while simplifying operation.

Another advantageous design of the spindle unit is achieved by the fact that the spindle rod can be driven rotationally by the driving rod, and the spindle rod may have an external thread being in engagement with an internal thread of a twist-proof spindle nut, and a spindle drive may be formed in this way. In this preferred specific embodiment, the spindle nut is fixedly connected to the plunger, so that the plunger can only execute a translational movement in the delivery of medication fluid. A translation movement of the plunger is to be preferred to a movement that is both rotational and translational because, in the latter case, seals arranged on the plunger can become twisted due to rotation of the plungers, so that the friction may be increased and the seal on the reservoir can no longer be ensured. For this advantageous specific embodiment, two possibilities are again conceivable for the filling. With the first arrangement, the plunger is in a retracted position at the beginning of the filling. Filling is accomplished manually by the user pushing the plunger forward by means of a pull-up rod in order to displace the air into the storage container. Then the user can move the plunger in reverse and draw medication fluid into the reservoir, wherein here again an adapter with an overflow needle can be used to establish the fluidic connection. Another advantageous and preferred specific embodiment, which is suitable in particular for filling, is formed by the fact that the plunger can be in an extracted position before the filling. In this specific embodiment, the spindle rod can be prevented from displacement in the delivery direction by an axial stop provided on the wall of the reservoir. In reverse rotation of the driving rod, the spindle rod may be supported on the wall of the reservoir, so that the plunger can move in reverse and medication fluid can flow from the supply container into the reservoir. By counting the motor steps in the reverse movement of the plunger by means of a control unit, the filling level, i.e., the amount of medication fluid in the reservoir can be calculated accurately after filling the reservoir. In a particularly advantageous embodiment, the spindle rod may be designed in two parts. Then the one part may be designed as a spindle rod driven by the driving rod and having an external thread. The second part may be designed as a disk, wherein the disk can absorb an axial force of the spindle rod on an inside radius in the reverse movement, and the disk can transfer the axial force on an outer radius to the wall of the reservoir. The disk may also be held in a twist-proof manner on the wall of the reservoir. In this preferred specific embodiment, the axial force may act on the disk at an inside diameter, so there is a reduced torque loss in reverse rotation due to friction generated between the spindle rod and the fixed disk. The preferred two-part specific embodiment of the spindle rod thus reduces the energy consumption in filling since the axial reaction force in filling is not absorbed directly by the wall of the reservoir but instead is absorbed by a stop formed on the disk and having a much smaller diameter than the diameter of the reservoir. The spindle rod is driven by the driving rod of the gear, wherein the spindle rod may have an elongated hole for axial accommodation of the driving rod. The elongated hole and the driving rod may be designed with profiling so that such a connection should only be able to transfer a drive torque.

A cylindrical design of the reservoir is preferred. In addition it is advantageous if the wall of the reservoir may be surrounded by an outside wall in the axial direction and if the outside wall may be fixedly connectable to a housing. The outside wall can ensure that a pressure from the outside acting on the outside wall cannot cause any unintentional dispensing of medication fluid due to deformation of the outside wall. The connection of the outside wall to the housing is preferably established by means of a bayonet connection. it is also advantage if the bayonet connection axially secures the reservoir in both the direction of advance and in the opposite direction. This ensures that the bayonet connection can absorb the axial force occurring when the reservoir is filled. The bayonet connection likewise absorbs the corresponding axial forces in the opposite direction during delivery.

The twist-proof connection of the spindle nut can be established by means of a tongue-and-groove connection between the reservoir and the spindle nut. Another advantageous embodiment of the twist-proof connection can also be created by the fact that the spindle nut may have radial wings, by means of which the spindle nut can be supported on an inside wall of the outside wall and the spindle nut can thereby be secured in a twist-proof manner. The outside wall may have an elliptical contour, i.e., not circular. The wings of the spindle nut protruding radially outward simplify the construction and assembly, wherein the function of the twist-proof lock of the spindle nut is by no means compromised. The twist-proof connection can also be established by the fact that protrusions on the spindle nut in the form of wings, for example, may engage in elongated slots formed on the reservoir wall.

The proposed metering device is particularly suitable for metering insulin in an insulin pump. Due to the properties of the metering device according to the invention, by which it can accurately dose and dispense very small amounts of medication fluid, the metering device is especially suitable for continuous subcutaneous infusion of insulin—CSII therapy—in children and young people. Extremely small metering increments of 0.0025 IU, for example, extremely low basal weights of 0.04 IU/h, for example, and extremely small bolus doses of 0.1 IU, for example, can be dispensed accurately by means of the metering device according to the invention. Due to the metering device according to the invention, the therapeutic result can be improved. Likewise everyday life for the user is simplified, dilution of the medication fluid to an insulin concentration of U100 may be omitted because the metering device according to the invention permits accurate metering of extremely small metering amounts and thus makes dilution no longer necessary in order to improve the accuracy of dispensing in the case of U100 insulin, for example. Use of the metering device according to the invention as an insulin pump is especially advantageous, in particular in use by children and young people.

The present invention also makes it possible for the reservoir to be fillable by the metering device itself. It is especially advantageous here that no axial force acts on the driving rod during filling. An axial reactor force is absorbed only by the bayonet connection and the housing. It is important for no axial tensile force to act on the driving rod during filling. In the prior art in which the plunger may be fixedly connected to the drive unit, such axial tensile forces can act on the spindle unit and the drive unit, so that reversal of the axial bearing play may take place.

The invention has surprisingly been able to show based on a theoretical derivation that only the sensitivity for the angle error can be influenced through the insulin concentration. Neither the sensitivity for the diameter error nor the sensitivity for the spindle pitch error can be influenced by the insulin concentration. This finding is novel. The present invention has also been able to show that optimization of the product of a spindle pitch and cross-sectional area of the reservoir has an effect on the metering accuracy equivalent to that of reducing the concentration. Furthermore, the invention has been able to show that the sensitivities for the equivalent diameter of the cross-sectional area and the spindle pitch are proportional to the respective inverse values of the diameter and the spindle pitch and these influence the accuracy in dispensing large amounts of medication fluid.

### BRIEF DESCRIPTION OF THE DRAWINGS

Exemplary embodiments of the invention are described below in greater detail on the basis of the drawings, in which:

FIG. 1a shows a first design range for an metering device according to the invention in comparison with the design ranges of the systems of the first and second generations,

FIG. 1b shows the diagram shown in FIG. 1a, wherein the design range for forming metering devices has been supplemented with a reduced sensitivity for the diameter error,

FIG. 1c shows a second design range for a metering device according to the invention in comparison with the design ranges of the systems of the first, second and third generations,

FIG. 2a shows a first exemplary embodiment of a reservoir having an integrated spindle unit and a pull-up rod in a starting condition in longitudinal section,

FIG. 2b shows the reservoir which is shown in FIG. 2a and is connected to a drive unit, to form a metering device according to the invention in longitudinal section,

FIG. 2c shows the reservoir illustrated in FIG. 2a in a view from beneath,

FIG. 3a shows a second exemplary embodiment of a metering device according to the invention in a starting state before filling in a longitudinal section,

FIG. 3b shows the metering device illustrated in

FIG. 3a, in a longitudinal section after filling,

FIG. 4a shows a reservoir with a spindle rod formed from two parts in a longitudinal section,

FIG. 4b shows the reservoir illustrated in FIG. 4a in cross section to the longitudinal axis,

FIG. 5a shows a third exemplary embodiment of a reservoir with an integrated spindle unit and a pull-up rod in a starting state in longitudinal section,

FIG. 5b shows the reservoir which is illustrated in FIG. 5a and is connected to a drive unit, for forming a metering device according to the invention in longitudinal section,

FIG. 6a shows a fourth exemplary embodiment of a metering device according to the invention in a starting status in longitudinal section before being filled and

FIG. 6b shows the metering device illustrated in FIG. 6a in a longitudinal section after being filled.

### METHODS FOR IMPLEMENTING THE INVENTION

The invention has taken as its goal to design a metering device such that extremely small metering amounts can be dispensed accurately and precisely. The basis for optimization of such a system is the law of error propagation according to Gauss which is described here in general in a first step and then is applied to a metering device. On the basis of the conclusions, which follow from this, finally a metering device can be optimized in such a way that it can yield an improved accuracy in dispensing extremely small metering amounts.

For optimization of the metering device consisting of a drive unit, a spindle unit driven by the drive unit and a reservoir for a medication fluid, wherein the reservoir has a wall and a plunger held in the wall, the law of error propagation according to Gauss is applied. This law is described by the following general equation for a function having three independent variables f(x₁, x₂, x₃):

$$Δ {f}^{2} = {( \frac{d f}{d {x}_{1}} )}^{2} \cdot Δ {x}_{1}^{2} + {( \frac{d f}{d {x}_{2}} )}^{2} \cdot Δ {x}_{2}^{2} + ( \frac{df}{d {x}_{3}} ) \cdot Δ {x}_{3}^{2}$$

The error of a function having three variables can thus be calculated by deriving the function according to the respective variables and adding that to their tolerance. The Gaussian approach says that one does not add up the derivations multiplied times their tolerance but instead one adds up their squares. A single error square therefore corresponds to a variance in which the three error variances are added. Finally, to obtain the error tolerance for the entire system, the root of the total error tolerance must be taken:

$$Δ f = \sqrt{{( \frac{df}{{dx}_{1}} )}^{2} \cdot Δ {x}_{1}^{2} + {( \frac{d f}{d {x}_{2}} )}^{2} \cdot Δ {x}_{2}^{2} + {( \frac{d f}{d {x}_{3}} )}^{2} \cdot Δ {x}_{3}^{2}}$$

The percentage influence of a single factor is obtained by dividing a single variance of a independent factor by the total variance:

$${error}_{i} = \frac{{( \frac{df}{{dx}_{i}} )}^{2} \cdot Δ {x}_{i}^{2}}{{( \frac{df}{{dx}_{1}} )}^{2} \cdot Δ {x}_{1}^{2} + {( \frac{d f}{d {x}_{2}} )}^{2} \cdot Δ {x}_{2}^{2} + {( \frac{d f}{{dx}_{3}} )}^{2} \cdot Δ {x}_{3}^{2}}$$

The derivations df/dxᵢ are referred to as sensitivities in mathematics.

On the basis of the theoretical principles just described, the Gaussian error propagation law will now be used. As the first step, the variable that is of interest and is to be analyzed is determined. In order to be able to analyze a metering device of the type described here, a relationship must be established between an output variable and independent input variables. For example, the transfer function from the motor angle to the metering amount can be determined for a metering device. If it is assumed that the motor angle is stepped down by a gear reduction ratio, and a spindle unit having a constant pitch is used, then in a first step the stroke of the spindle may be determined as a function of the motor angle:

$$Δ h = \frac{θ}{i} \cdot p$$

where Δh is given in mm of the stroke, θ is the motor angle in angular degrees, i is the gear reduction ratio and p is the pitch of the spindle in mm/angular degree. By multiplying the spindle pitch times a cross-sectional area Q of a reservoir in mm², the volume delivered is obtained as a function of the motor angle:

$$Δ V = \frac{θ}{i} \cdot p \cdot Q$$

Finally, the volume can be multiplied times a factor for the concentration, so that ultimately the dose administered ΔU can be determined. For example, this factor amounts to 0.1 for U100 insulin, i.e., a volume of 10 mm³ must be multiplied times a factor of 0.1 to thereby determine the amount of insulin units dispensed in IU. A volume of 10 mm³ therefore contains a 1 IU of insulin at a concentration U100 insulin; Cᵢₙₛᵤₗᵢₙ is the factor for conversion from 1 volume of insulin to an amount of insulin in units of IU (international units).

$$Δ U = \frac{θ}{i} \cdot p \cdot Q \cdot {C}_{insulin}$$

In the following equation, the relationship between the metering amount of insulin and the independent variables is represented, wherein the cross-sectional area Q has been replaced by the corresponding area equation π·Dᵢ²/4. The figures show only the diameter Dᵢ, from which the cross-sectional area Q can be determined. The Gaussian error propagation law will now be applied to the following function, which describes the relationship between the metering amount of medication fluid dispensed to the motor angle of a metering device:

$$Δ U = \frac{θ}{i} \cdot p \cdot \frac{π \cdot {D}_{i}^{2}}{4} {C}_{insulin}$$

The metering amount dispensed ΔU of IU insulin can be derived according to the independent variables. The latter include the pitch p, the equivalent diameter of the reservoir Dᵢ and the motor angle θ. The motor angle error, when considered more broadly, may be any error angle of the drive unit, i.e., an angle error that is caused by the components motor and gear as a result of friction or tolerance errors in the gear wheels, for example. Accordingly, the angle error may be considered as any error in which a predetermined angle of a control unit cannot be converted correctly by the drive unit. At the output of the gear, an angle error for the spindle unit is therefore the result. Due to the derivation of the function according to the independent variables, this yields the sensitivities. The Gaussian error propagation law can be applied when the independent variables are independent of one another and have a normal distribution. It is assumed here that these conditions are met. The three sensitivities are shown below:

Sensitivity for the equivalent diameter Dᵢ:

$$\frac{d Δ U}{d {D}_{i}} = p \cdot \frac{π \cdot {D}_{i}}{2} \cdot θ \cdot \frac{{C}_{insulin}}{i}$$

Sensitivity for the pitch p:

$$\frac{d Δ U}{d p} = \frac{π \cdot {D}_{i}^{2}}{4} \cdot θ \cdot \frac{{C}_{insulin}}{i}$$

Sensitivity for the motor angle θ:

$$\frac{d Δ U}{d θ} = p \cdot \frac{π \cdot {D}_{i}^{2}}{4} \cdot \frac{{C}_{insulin}}{i}$$

The result for the motor angle is especially interesting. The sensitivity for the motor angle is proportional to the product of the spindle pitch on the cross-sectional area of the reservoir. In order for a metering error due to inaccurate positioning of the motor to be reducible, the product of the spindle pitch on the cross-sectional area of the reservoir must be minimized. Thus the two factors—spindle pitch and cross-sectional area—must be kept as small as possible. The sensitivities derived by the preceding method can be further simplified by describing the angle θ as a function of the amount of insulin ΔU to be dispensed:

$$θ = \frac{Δ U \cdot i}{p \cdot ( \frac{π \cdot {D}_{i}^{2}}{4} ) {C}_{insulin}}$$

Now this yields the following simplified presentations for the sensitivities, which are new.

Sensitivity for the equivalent diameter Dᵢ:

$$\frac{d Δ U}{{dD}_{i}} = \frac{2 \cdot Δ U}{{D}_{i}}$$

Sensitivity for the pitch p:

$$\frac{d Δ U}{dp} = \frac{Δ U}{p}$$

Sensitivity for the motor angle θ:

$$\frac{d Δ U}{d θ} = p \cdot \frac{π \cdot {D}_{i}^{2}}{4} \cdot \frac{{C}_{insulin}}{i}$$

It must be pointed out here that the sensitivity for the diameter as well as the sensitivity for the spindle pitch cannot be influenced by the insulin concentration Cᵢₙₛᵤₗᵢₙ. This means that dilution of insulin from U100 insulin, for example, to U50 insulin does not lead to an improvement with regard to the dispensing accuracy for the diameter error and the spindle pitch error. This is a new finding. The insulin concentration has only a direct influence on the sensitivity of the motor angle error which can be influenced directly by the insulin concentration so that, for example, the error can be reduced by one-half by using U50 insulin in comparison with U100 insulin. The dispensing accuracy can be improved not only by diluting the concentration of the insulin but according to the sensitivity for the angle error, the dispensing accuracy can be improved significantly beyond an optimized value of the diameter and spindle pitch parameters. This is because the product of the spindle pitch and the area

$$p \cdot \frac{π \cdot {D}_{i}^{2}}{4}$$

is directly proportional to the sensitivity of the motor angle error, like the insulin concentration. Due to an inventive choice of said multiplication product, a metering device can be created which can dispense extremely small metering amounts in particular with precision so that even insulins in a concentration of U100 can be administered with greater precision than is possible in the state of the art today. Furthermore, the following relationship is apparent from the sensitivities given above. An excessive reduction in the spindle pitch p or the diameter Dᵢ causes an unfortunate increase in the respective sensitivity, which is a disadvantage. For example, if the reservoir diameter Dᵢ is reduced from 10 mm to 5 mm, the corresponding sensitivity is doubled. At the same tolerance for the diameter, this yields larger errors in dispensing. Both the sensitivity for the diameter and the sensitivity for the pitch are indirectly proportional to the diameter and the spindle pitch, respectively. In particular, the systems of the third generation have very small diameters for the metering cylinder, so that there is an unfortunate increase in the corresponding diameter error in dispensing. For the total dispensing error dΔU which takes into account the error square of diameter, spindle pitch and angle error, this therefore yields the following equations:

$$d Δ {U}^{2} = {( \frac{2 \cdot Δ U}{{D}_{i}} )}^{2} \cdot Δ {D}_{i}^{2} + {( \frac{Δ U}{p} )}^{2} \cdot Δ {p}^{2} + {( p \cdot \frac{π \cdot {D}_{i}^{2}}{4} \cdot \frac{{C}_{insulin}}{i} )}^{2} \cdot Δ {θ}^{2}$$

$$d Δ U = \sqrt{{( \frac{2 \cdot Δ U}{{D}_{i}} )}^{2} \cdot {AD}_{i}^{2} + {( \frac{Δ U}{p} )}^{2} \cdot Δ {{p}^{2} ( p \cdot \frac{π \cdot {D}_{i}^{2}}{4} \cdot \frac{{C}_{insulin}}{i} )}^{2} \cdot Δ {θ}^{2}}$$

The results for the procedure described above for the interpretation of a metering device are summarized below. These results are listed in the following Table 3 for a system of the first generation, a system of the second generation and a system of the third generation as well as a system according to the invention. Since U100 insulin is used in insulin pump therapy in general, and this insulin concentration has become established as the gold standard, the following calculations are therefore based on this conventional concentration. In general the findings of the present invention are also applicable to other insulin concentrations such as U40 insulin, for example.

TABLE 3

| Metering amount in IU U100, ΔU | | | | |
|---|---|---|---|---|
| 0.0025 | 0.05 | 0.1 | 1 | 10 |

First generation pump

| | | | | | |
|---|---|---|---|---|---|
| Metering error± in IU U100, dΔU | 0.00499 | 0.00504 | 0.00516 | 0.01394 | 0.13027 |
| Metering error± in % of metering amount | 199.73% | 10.07% | 5.16% | 1.39% | 1.30% |
| Reservoir diameter error in % | 0.00% | 0.68% | 2.61% | 35.73% | 40.92% |
| Spindle pitch error in % | 0.00% | 0.99% | 3.76% | 51.45% | 58.93% |
| Motor angle error in % | 100.00% | 98.33% | 93.64% | 12.83% | 0.15% |

Second generation pump

| | | | | | |
|---|---|---|---|---|---|
| Metering error± in IU U100, dΔU | 0.00453 | 0.00457 | 0.00471 | 0.01354 | 0.12769 |
| Metering error± in % of metering amount | 181.16% | 9.15% | 4.71% | 1.35% | 1.28% |
| Reservoir diameter error in % | 0.00% | 0.75% | 2.84% | 34.27% | 38.54% |
| Spindle pitch error in % | 0.00% | 1.20% | 4.52% | 54.54% | 61.34% |
| Motor angle error in % | 100.00% | 98.05% | 92.65% | 11.19% | 0.13% |

Third generation pump

| | | | | | |
|---|---|---|---|---|---|
| Metering error± in IU U100, dΔU | 0.00029 | 0.00125 | 0.00245 | 0.02437 | 0.24369 |
| Metering error± in % of metering amount | 11.78% | 2.50% | 2.45% | 2.44% | 2.44% |
| Reservoir diameter error in % | 3.56% | 78.76% | 82.01% | 83.15% | 83.16% |
| Spindle pitch error in % | 0.72% | 15.95% | 16.61% | 16.84% | 16.84% |
| Motor angle error in % | 95.72% | 5.30% | 1.38% | 0.01% | 0.00% |

Invention - MeaPump Juvenile

| | | | | | |
|---|---|---|---|---|---|
| Metering error± in IU U100, dΔU | 0.00074 | 0.00113 | 0.00186 | 0.01713 | 0.17115 |
| Metering error± in % of metering amount | 29.55% | 2.26% | 1.86% | 1.71% | 1.71% |
| Reservoir diameter error in % | 0.22% | 37.79% | 55.54% | 65.74% | 65.86% |
| Spindle pitch error in % | 0.11% | 19.59% | 28.79% | 34.08% | 34.14% |
| Motor angle error in % | 99.66% | 42.63% | 15.66% | 0.19% | 0.00% |

Table 4 also lists the tolerances used for the calculation. The error square of the independent factors and the total errors in units of IU insulin have been summarized in Table 4. The tolerance for the spindle pitch is ±1% of the pitch. The tolerance for the diameter is ±0.05 mm for all diameters. The tolerances used corresponds to today's production tolerances.

TABLE 4

| | Metering amount in IU U100, ΔU | | | | | | |
|---|---|---|---|---|---|---|---|
| | | | 0.0025 | 0.05 | 0.1 | 1 | 10 |
| | Value | Tol.± | Error² | Error² | Error² | Error² | Error² |
| First generation pump | | | | | | | |
| Gear reduction ratio | 906 | | | | | | |
| Reservoir diameter in mm | 12.00 | 0.05 | 4.34⁻¹⁰ | 1.74⁻⁰⁷ | 6.94⁻⁰⁷ | 6.94⁻⁰⁵ | 6.94⁻⁰³ |
| Pitch in mm/revolution | 1.20 | 0.012 | 6.25⁻¹⁰ | 2.50⁻⁰⁷ | 1.00⁻⁰⁶ | 1.00⁻⁰⁴ | 1.00⁻⁰² |
| Motor angle in angular degree | | 120 | 2.49⁻⁰⁵ | 2.49⁻⁰⁵ | 2.49⁻⁰⁵ | 2.49⁻⁰⁵ | 2.49⁻⁰⁵ |
| Insulin concentration U100 | 0.10 | | | | | | |
| Error in IU total, dΔU | | | 0.00499 | 0.00504 | 0.00516 | 0.01394 | 0.13027 |
| Second generation pump | | | | | | | |
| Gear reduction ratio | 920 | | | | | | |
| Reservoir diameter in mm | 12.62 | 0.05 | 3.93⁻¹⁰ | 1.57⁻⁰⁷ | 6.28⁻⁰⁷ | 6.28⁻⁰⁵ | 6.28⁻⁰³ |
| Pitch in mm/revolution | 1.00 | 0.010 | 6.25⁻¹⁰ | 2.50⁻⁰⁷ | 1.00⁻⁰⁶ | 1.00⁻⁰⁴ | 1.00⁻⁰² |
| Motor angle in angular degree | | 120 | 2.05⁻⁰⁵ | 2.05⁻⁰⁵ | 2.05⁻⁰⁵ | 2.05⁻⁰⁵ | 2.05⁻⁰⁵ |
| Insulin concentration U100 | 0.10 | | | | | | |
| Error in IU total, dΔU | | | 0.00453 | 0.00457 | 0.00471 | 0.01354 | 0.12769 |
| Third generation pump | | | | | | | |
| Gear reduction ratio | 920 | | | | | | |
| Reservoir diameter in mm | 4.50 | 0.05 | 3.09⁻⁰⁹ | 1.23⁻⁰⁶ | 4.94⁻⁰⁶ | 4.94⁻⁰⁴ | 4.94⁻⁰² |
| Pitch in mm/revolution | 0.5 | 0.005 | 6.25⁻¹⁰ | 2.50⁻⁰⁷ | 1.00⁻⁰⁶ | 1.00⁻⁰⁴ | 1.00⁻⁰² |
| Motor angle in angular degree | | 120 | 8.30⁻⁰⁸ | 8.30⁻⁰⁸ | 8.30⁻⁰⁸ | 8.30⁻⁰⁸ | 8.30⁻⁰⁸ |
| Insulin concentration U100 | 0.10 | | | | | | |
| Error in IU total, dΔU | | | 0.00029 | 0.00125 | 0.00245 | 0.02437 | 0.24369 |
| Inventive MeaPump Juvenile | | | | | | | |
| Gear reduction ratio | 920 | | | | | | |
| Reservoir diameter in mm | 7.20 | 0.05 | 1.21⁻⁰⁹ | 4.82⁻⁰⁷ | 1.93⁻⁰⁶ | 1.93⁻⁰⁴ | 1.93⁻⁰² |
| Pitch in mm/revolution | 0.5 | 0.005 | 6.25⁻¹⁰ | 2.50⁻⁰⁷ | 1.00⁻⁰⁶ | 1.00⁻⁰⁴ | 1.00⁻⁰² |
| Motor angle in angular degree | | 120 | 5.44⁻⁰⁷ | 5.44⁻⁰⁷ | 5.44⁻⁰⁷ | 5.44⁻⁰⁷ | 5.44⁻⁰⁷ |
| Insulin concentration U100 | 0.10 | | | | | | |
| Error in IU total, dΔU | | | 0.00074 | 0.00113 | 0.00186 | 0.01713 | 0.17115 |

Extension conclusions can be drawn from the preceding calculations and results. The metering devices of the first and second generations have the least error for large dispensing amounts because of the lowest sensitivities for the diameter and the spindle pitch. The third-generation system has the largest error for the large dispensing amounts. The third-generation system has the greatest sensitivity for the diameter. In dispensing large dosage amounts, therefore third-generation systems have a large dispensing error. The sensitivity for the spindle pitch is proportional to the inversion of the pitch. A reduction in the spindle pitch at the same tolerance therefore leads to an increase in the corresponding error square. Miniaturization of the parameters, as may be the case in particular with the third-generation systems, increases the sensitivity. At the same tolerances, the error squares of the diameter and spindle pitch show an unfavorable increase for third-generation systems. However, third-generation systems have the best dispensing accuracy for the smallest metering increment of 0.0025 IU. At a dispensing rate of 0.05 IU/h which is relevant for CSII treatment of children and young people, however, the metering device according to the invention has a better accuracy in comparison with the third-generation systems as well as in comparison with the first and second-generation systems. The metering device according to the invention has similar or better dispensing accuracies in the range of the smallest metering amounts in comparison with a system of the third generation. In the range of large metering amounts, it has only a marginally worse performance in comparison with the first and second-generation systems. The metering device according to the invention can dispense extremely small metering amounts accurately and, in doing so, achieves or even exceeds the performance of the third-generation systems. However, the metering device according to the invention also has an error at large metering amounts which is insignificantly greater than that of the first- and second-generation systems. At moderate rates, the system according to the invention turns out much better than the systems known from the prior art. At a metering rate of 0.1 IU/h, the system according to the invention can deliver the total metering amount with an accuracy of ±1.86% after a dispensing interval of 1 hour. A system of the first generation or second generation achieves an error tolerance of only ±5.16% or ±4.71%, respectively, whereas the third-generation system has an error of ±2.45%. Moreover, it must be pointed out here that for a dose of 0.05 IU/h, the error for the system according to the invention amounts to 2.26% after 1 hours. For a first-generation system, this error amounts to 10.07%, which is substantial. In order for the system of the first generation to have a comparable error, the insulin must be diluted in 1/5 ratio, i.e., U20 insulin must be used. This comparison shows how much a metering device according to the invention, in which the product p·π·Dᵢ²/4=p·Q, which is formed from the spindle pitch and the reservoir area, is selected to be optimal, is capable of improving the dispensing accuracy in comparison with the prior art. The following table compares the reservoir area Q and the spindle pitch p for the systems of the first and second generations, one system of the third generation and a system according to the invention (MeaPump Juvenile). The product of the cross-sectional area and the pitch can be formed from these parameters. Likewise the sensitivity of the diameter which is proportional to the inverse of the diameter is also shown.

Table 5 shows that the products formed form the spindle pitch and the area are much smaller for the system according to the invention and the third-generation system than those for the first- and second-generation systems. However, the sensitivity for the dispensing error of the diameter which is proportional to the inverse of the diameter (1/Dᵢ), is largest for the third-generation system at 0.222. As described above, the result is that a large dispensing error must be expected with large metering amounts.

TABLE 5

| | | Pitch p in | | |
|---|---|---|---|---|
| | Area Q | mm²/angular | Product | |
| | in mm² | degree | p*Q | 1/Dᵢ |
| First generation | | | | |
| Roche Accuchek Spirit | 113.10 | 0.0034 | 0.377 | 0.083 |
| Roche D-Tron | 67.20 | 0.0034 | 0.224 | 0.108 |
| Minimed, Paradigm Veo | 113.10 | 0.0028 | 0.314 | 0.083 |
| Animas, Ping, 2020 | 95.03 | 0.0028 | 0.264 | 0.091 |
| Deltec, Cozmo | 113.10 | 0.0028 | 0.314 | 0.083 |
| Second generation | | | | |
| Insulet, Omnipod | 125.00 | 0.0011 | 0.139 | 0.079 |
| MeaPump Adult | 125.00 | 0.0028 | 0.347 | 0.079 |
| Third generation | | | | |
| System of third generation | 15.90 | 0.0014 | 0.022 | 0.222 |
| System according to invention | | | | |
| MeaPump Juvenile (invention) | 40.72 | 0.0014 | 0.057 | 0.139 |

The metering device according to the invention is capable of dispensing small metering amounts with a very high precision, wherein the error of the motor angle is minimized for small metering amounts. Likewise, the metering device according to the invention is capable of dispensing large amounts of medication fluid with a very high precision. In the case of large metering amounts, the percentage error of the motor angle tends toward zero (the absolute value remains constant), while the error of the diameter due to diameter tolerance and the error of the spindle pitch due to pitch tolerances are significant here. The system according to the invention can thus dispense extremely small metering amounts with a much greater accuracy than in the prior art consisting of systems of the first and second generations.

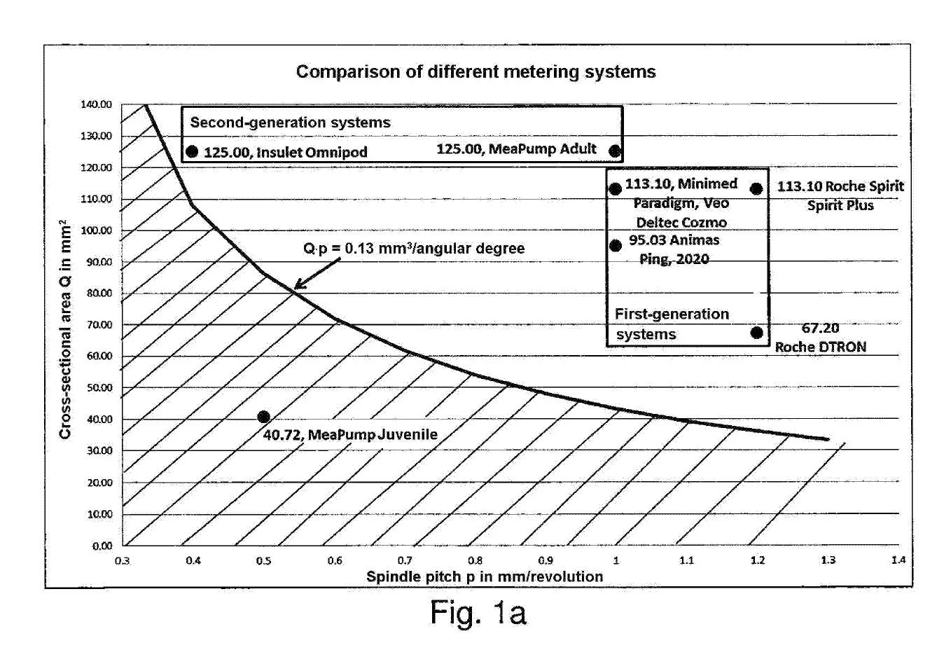

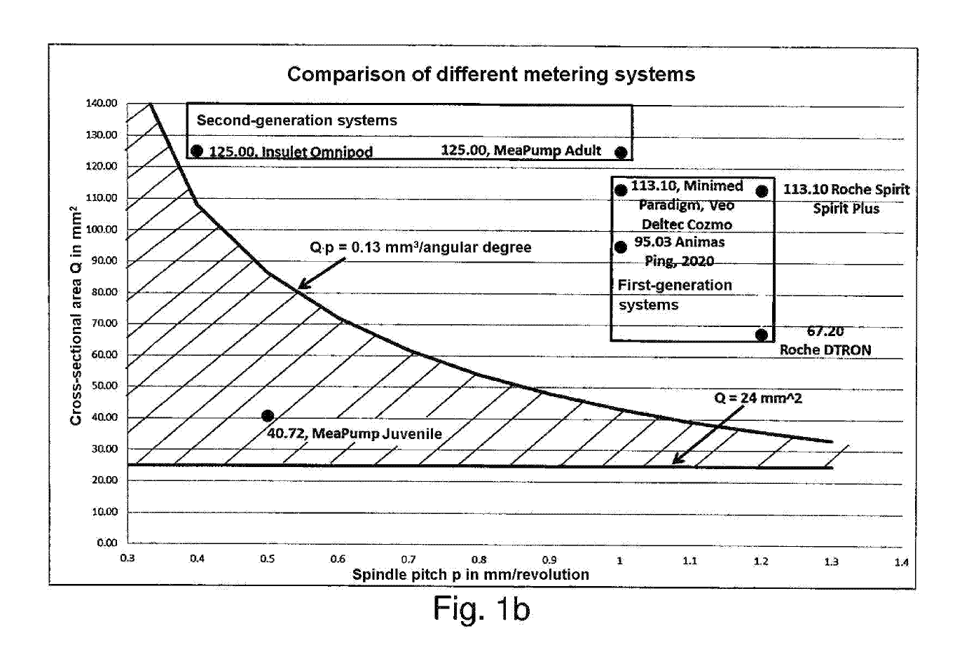

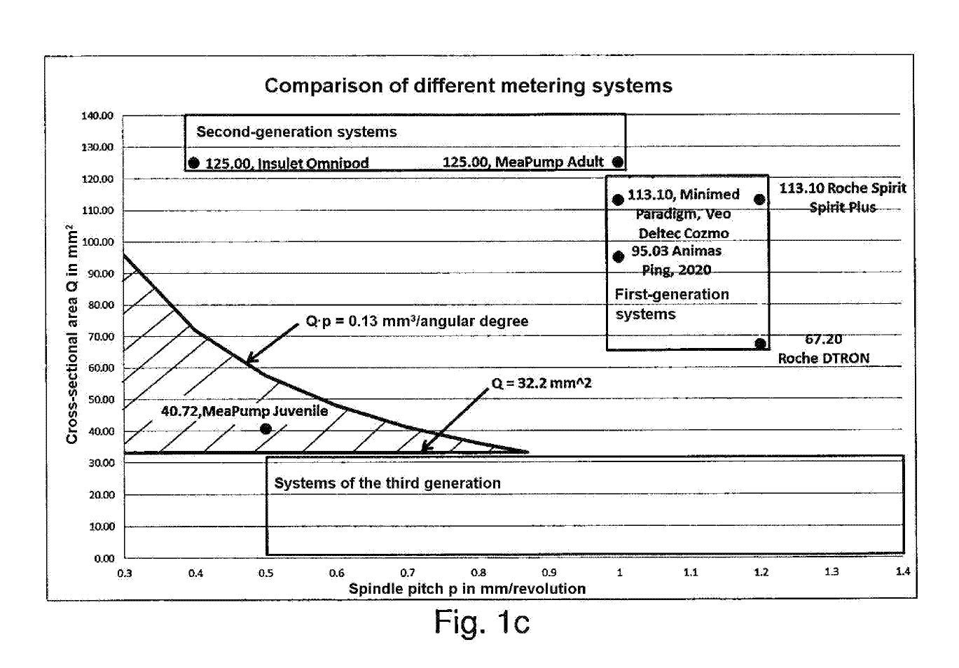

The results of Table 5 above have been plotted graphically in FIG. 1a where the pitch of the spindle is plotted on the x axis and the cross-sectional area Q is plotted on the y axis. The respective systems of the first generation and the second generation have been combined in graphical quadrants, wherein the respective cross-sectional areas are given in mm². It is claimed according to the invention that the product formed form the cross-sectional area and the pitch should be less than 0.13 mm³/angular degree. The limiting line for this condition can thus be determined with the following equation:

$$Q = \frac{0.13 ( \frac{{mm}^{3}}{angular degree} ) 360 ( \frac{angular degree}{revolution} )}{p ( mm / revolution )}$$

The average product of the spindle pitch and the cross-sectional area amounts to 0.30 mm³/angular degree for systems of the first generation. The metering device according to the invention has an equivalent dilution of the concentration of more than 50% with a product of the spindle pitch and the cross-sectional area amounting to less than 0.13=³/angular degree. This means that first-generation systems must use an insulin concentration lower than U50 to achieve at least the same dispensing accuracy as that of the metering device according to the invention. The lower limit according to claim 3 amounts to 24 mm², which corresponds to an equivalent diameter of 5.5 mm. The lower limit ensures that the sensitivity for the diameter does not exceed a maximum value and therefore accurate dispensing of large metering amounts can be ensured, whereas the upper limit for the claimed design range ensures that small and extremely small metering amounts can be dispensed accurately. The range claimed in FIG. 1b has good properties with regard to dispensing accuracy over the entire metering range. This range is especially suitable for CSII treatment of children and young people with type 1 diabetes, for whom undiluted U100 insulin can be used. FIG. 1c shows another restricted range of the invention. In this range, it is possible to create metering devices having a further improvement in dispensing accuracy. The range shown in FIG. 1c has a cross-sectional area greater than 32.2 mm², which corresponds to a diameter of 6.4 mm for the reservoir. The product of the cross-sectional area and the pitch is represented by the upper limiting line. This limit satisfies the equation Q·p=0.08 mm³/angular degree. FIG. 1c also shows the third-generation device. The third-generation systems have a new design with a reservoir and a separate metering cylinder. These systems are shown here only for the sake of thoroughness. Only the systems of the first and second generation as well as the embodiment according to the invention belong to the same category, in which the reservoir itself has a movable plunger for dispensing medication fluid. Metering devices of the first and second generation as well as the invention could be considered as injection pumps, for which a plunger of a reservoir is displaced by a drive unit and medication fluid is delivered in this way.

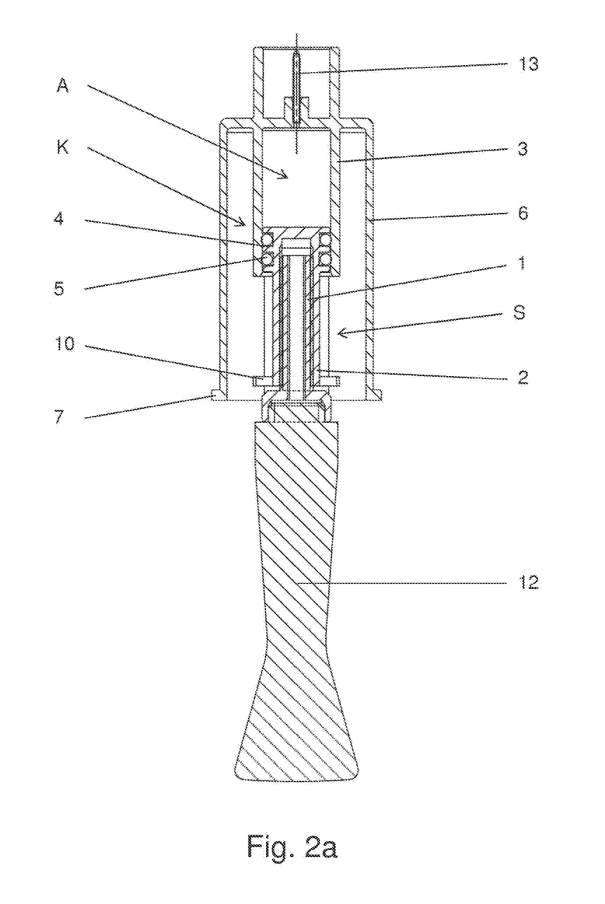

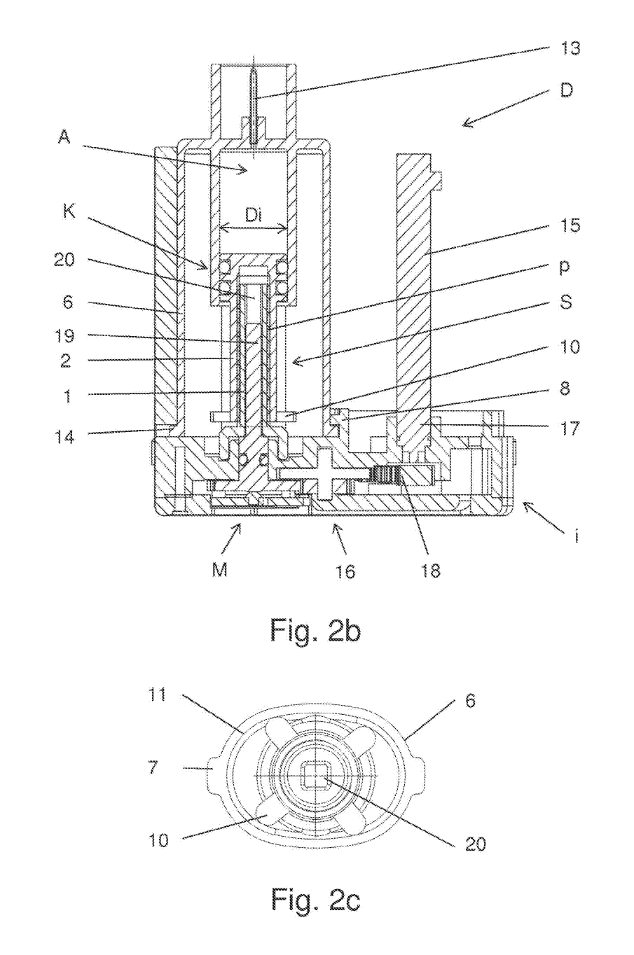

FIG. 2a shows a reservoir A which has an integrated spindle unit S. The spindle unit S consists of a spindle rod 1 and a spindle nut 2, wherein the spindle nut 2 is movable in the embodiment shown here. The reservoir A has an inside wall 3, along which a plunger K is guided. The plunger K itself is connected directly to the spindle nut 2. Seals 4, which are designed here in the form of O-rings 5, are provided between the inside wall 3 and the plunger K. In addition to the inside wall 3, which has an inside diameter of 7.2 mm here, the reservoir A also has an outside wall 6. Inside wall 3 and outside wall 6 are fixedly connected to one another. The outside wall 6 also has locking cams 7, by means of which the reservoir A can be connected to a fixed housing 8. Therefore, in the forward movement of the spindle nut 2 and/or the plunger K, the spindle nut cannot move rotationally, i.e., it forms a twist-proof lock. In the exemplary embodiment of FIGS. 2a and 2b shown here, the spindle nut is designed in the form of a radial wing 10. The radial wing 10 is shown in a view from beneath in FIG. 2c. The outside wall 6 is designed with an oval shape. The wings 10 protruding radially outward are supported on an inside wall 11 of the outside wall 6 and thus prevent a rotational movement of the spindle nut 2. In the starting condition shown in FIG. 2a, the spindle nut 2 is shown in its retracted position, wherein the spindle rod 2 may be connected to a pull-up rod 12. To fill the reservoir A with insulin, the user first connects the reservoir to a storage container by means of an adapter. The reservoir A itself has a connecting needle 13 by means of which the fluidic connection to the reservoir A is established by means of the adapter. In a first handling step, the user pushes the plunger K in the forward direction and thereby displaces the air in the reservoir A into the supply container. In another handling step, there is a plunger movement in the opposite direction, wherein insulin can then flow overflow from the supply container into the reservoir A. FIG. 2b shows a metering device D according to the invention. The reservoir A having the integrated spindle unit S is connected to a drive unit M after being filled, so that the metering device D is formed. By means of a bayonet connection 14, the reservoir A is connected to a fixed housing 8. A motor 15 of the drive unit M drives an driving rod 19, so that it can rotate by means of a gear 16, wherein a planetary gear 17 and a deflecting gear 18 are present here. The total gear reduction ratio i consists of the gear reduction ratio of the planetary gear 17 and the gear reduction ratio of the deflecting gear 18 in the form of a spur gear. The motor has Hall sensors for the positioning, these sensors having a motor increment of 60 angular degrees of the motor angle. The exemplary embodiment shown in FIG. 2b has a gear reduction ratio i of 920, a spindle pitch p of 0.5 mm/revolution and a diameter Dᵢ of 7.2 mm. This yields the data according to the invention, as depicted in Table 5, according to which the product of the cross-sectional area and the pitch amounts to 0.057 mm³/angular degree, and the sensitivity for the diameter is proportional to 0.139 mm⁻¹. The driving rod 19, which is driven to rotate engages in a profiled elongated hole 20 which is formed on the spindle rod 1. In rotation of the driving rod 19, it entrains the spindle rod 1. The spindle nut 2 which is secured in a twist-proof manner strikes the spindle rod 1 and thus moves in the axial direction of advance. Due to the displacement of the plunger K, medication fluid is dispensed to the user. The fluid path downstream from the connecting needle 13 is not shown in FIG. 2b. In general, the fluid path has a fluidic connection from the connecting needle 13 to the subcutaneous tissue of a user. A cannula leading into the subcutaneous tissue can ensure the connection of the user to the metering device. The double-walled design of the reservoir A shown in FIGS. 2a to 2c has various advantages. First the outside wall 6 in an oval shape can secure the twist-proof locking of the spindle nut 2. Furthermore, the outside wall 6 protects the actual reservoir A from impacts and the like. Pressure on the outside wall 6 does not result in any compression of the reservoir cylinder in the interior. Such a double-walled reservoir A does not require a fixed housing, such as that provided in metering devices of the first generation, for example, in order to protect the ampoules.

The example in FIGS. 2a to 2c can also be embodied with an alternative twist-proof lock. The twist-proof lock may involve a tongue-and-groove connection between the inside wall and the spindle nut. In this variant, the inside wall is lengthened toward the drive unit. A pull-up rod is suitable for filling in this variant.

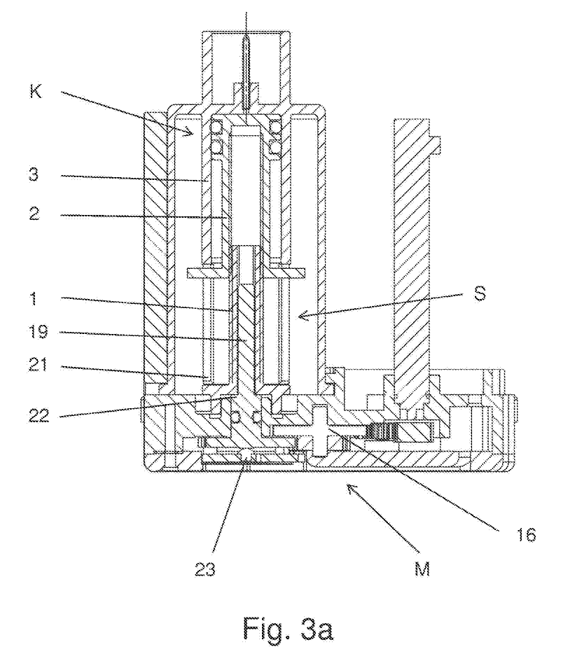

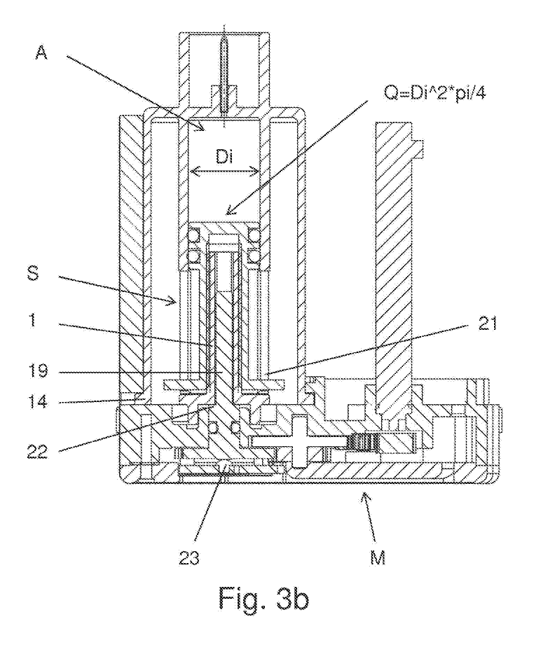

Another exemplary embodiment is depicted in FIGS. 3a and 3b. This exemplary embodiment has a design similar to that in the embodiment of FIGS. 2a to 2c because, here again, a spindle rod 1 is driven to rotate and a twist-proof-locking spindle nut 2 executes a linear movement. In a starting state before filling in FIG. 3a, the spindle nut and the plunger K connected to it are in an upper position. Due to a reverse rotation of the spindle nut 2, the spindle nut 2 moves in reverse and draws insulin out of a storage container and into the reservoir A and thereby fills the reservoir A. The exemplary embodiment in FIGS. 3a and 3b therefore has the advantage that the filling need not be carried out by hand but instead can be done automatically by the metering device. In order for this to be possible, the spindle rod 1 must be supportable during the reverse movement of the plunger K. This support is advantageously provided on the inside wall 3. The wall 3 therefore has a stop 21 for the spindle rod 1. In the reverse rotation of the spindle rod 1, it is supported on the wall 3. After the filling, the drive unit changes its direction of rotation. By rotating the spindle rod 1 in the forward direction, the spindle rod 1 moves against a stop 22 formed on the driving rod 19. In the forward direction the spindle rod 1 is thus supported on the driving rod 19. In filling the reservoir A however the spindle rod 1 is supported on the inside wall 3 of the reservoir A. The twist-proof locking of the spindle nut 2 may take place by means of longitudinal grooves and cams between the inside wall and the spindle nut 2. The exemplary embodiment in FIGS. 3a and 3b therefore has the advantage that the reservoir A of the drive unit M can be filled itself so this can spare the user the tedious job of filling. Furthermore the support of the spindle rod S on the inside wall 3 during filling can be very advantageous because this makes it possible to ensure that no axial forces are acting on the driving rod 19 during the filling and that the drive unit M is being guides. Only the bayonet connection 14, which is provided for such forces is claimed for the forces that act during filling and the following dispensing of the medication fluid. Filling by way of the drive unit M is additionally advantageous in that a control unit can calculate the filling volume on the basis of the reverse motor steps. It is therefore possible to fill the reservoir A with a certain volume precisely as intended by the user. After the filling, the spindle rod 1 runs up against the stop 22 formed on the driving rod 19. Beneath the driving rod 19 there is a force sensor 23 which can monitor an axial force acting on the spindle unit S during conveyance. The force senor 23 primarily has the task of detecting occlusions. It can likewise detect that the spindle rod 1 has run up against the stop 22 formed on the driving rod 19.

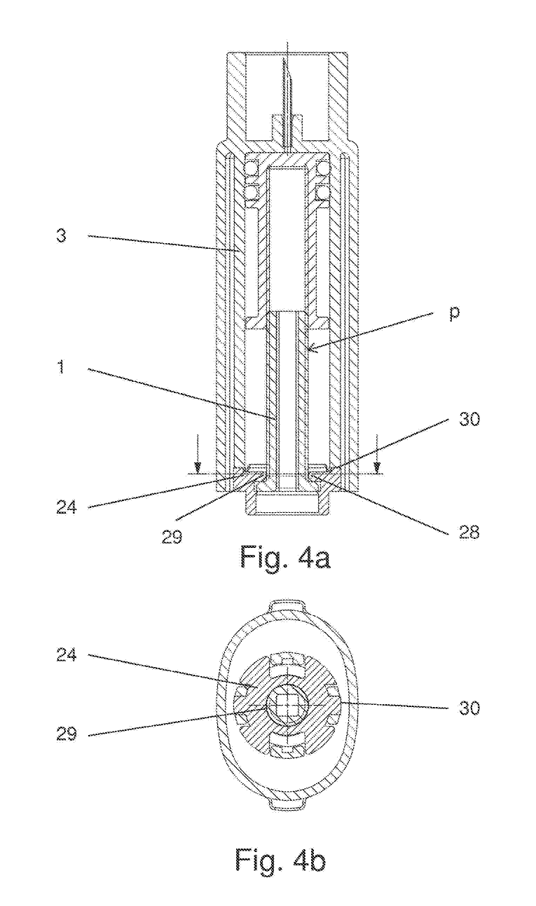

FIG. 4a shows a reservoir A similar to the design shown in FIGS. 3a and 3b. The reservoir A in FIG. 4a differs in that the spindle rod 1 is designed in two parts here. In addition to the spindle rod 1, FIG. 4a also shows a stop disk 24, which is fixedly connected to the inside wall 3. The stop disk 24 has a stop 28 for the spindle rod 1, wherein the active radius 29 for support of the spindle rod on the stop disk 24 can be reduced. The torque loss generated between the spindle rod 1 and its axial stop 28 can thus be greatly reduced. The reservoir A illustrated in FIG. 4a thus has the advantage that the torque losses occurring in operation in reverse can be reduced by providing the support between the spindle rod 1 and its stop 28 formed on the stop disk on a smaller radius 29. The axial force is transferred from an outer radius 30 to the inside wall 3 by way of the stop disk. The stop disk 24 is connected to the inside wall 3 in a rotationally fixed and axially secured manner. FIG. 4b shows a view across the longitudinal axis in which the two-piece design of the spindle rod 1 is readily visible.

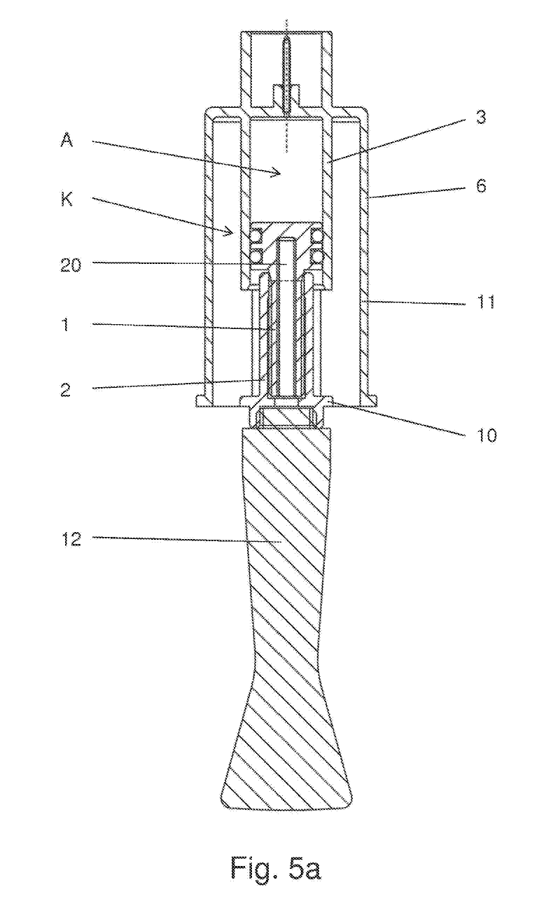

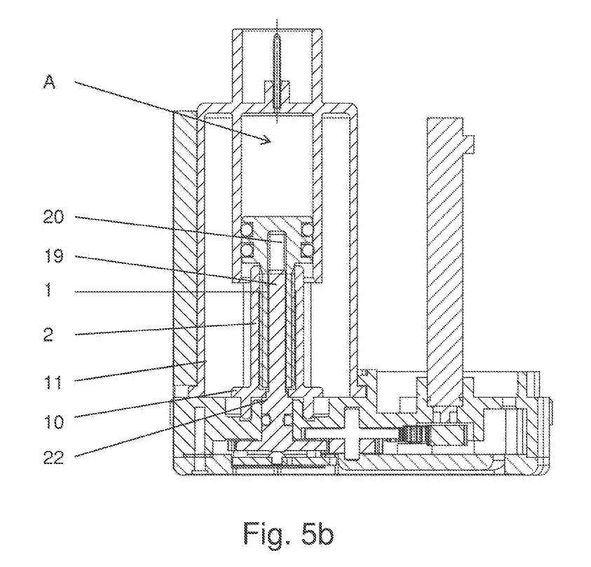

FIGS. 5a and 5b show another exemplary embodiment of the convenient filling method. In this example, the plunger K is connected to the spindle rod 1. The spindle nut 2 is locked in a twist-proof manner and can be supported during a forward motion on a stop 22 formed on the driving rod 19. The spindle rod 1 in turn has an elongated hole 20, in which the driving rod 19 can engage. In contrast with the embodiments discussed previously, the spindle rod 1 here executes a rotation created by the driving rod 19 as well as a linear movement provoked by the twist-proof locking of the spindle nut 2. The spindle nut 2 may be connected to a pull-up; rod 12 in order to perform the filling of the reservoir A in a convenient manner. In this case, a radial wing 10 is in turn suitable as an element for the twist-proof locking, which can be supported on the inside wall 11.

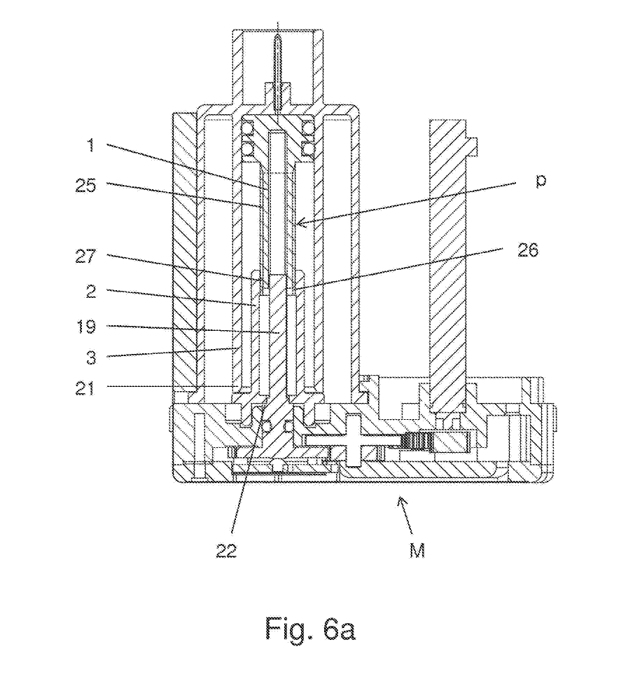

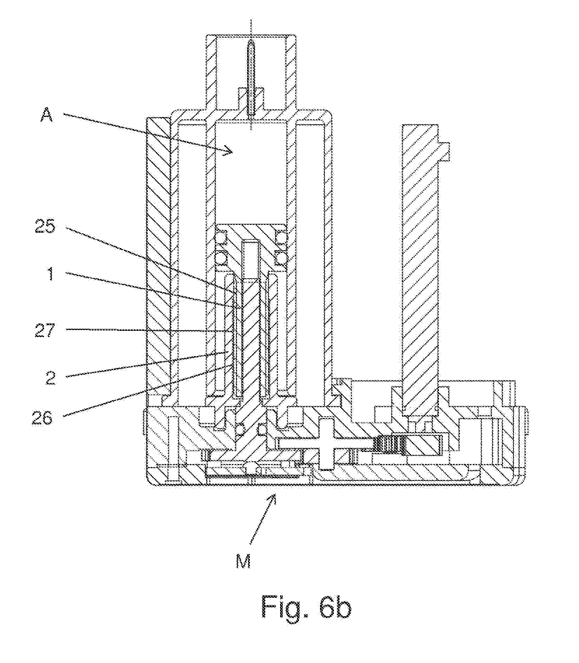

FIGS. 6a and 6b show another exemplary embodiment which is provided for automatic filling. The spindle nut 2, which is locked in a twist-proof position, is supported on the inside wall 3 on the stop 21 of the reservoir during filling. For example, profiling is used for the twist-proof locking between the spindle nut 2 and the inside wall 3. Simple tongue-and-groove connections between the components are also conceivable and should prevent rotation of the spindle nut 2. Axial displacement of the spindle nut 2 is prevented by the stop 21 during reverse rotation, and in the opposite direction the spindle nut 2 strikes against its axial stop 22 formed on the driving rod 19.

In the exemplary embodiments, the spindle rod 1 has an external thread 25 and the spindle nut 22 has an internal thread 26. The external thread and internal thread together thus form a spindle drive 27. It should be pointed out that a reversal of the spindle nut 2 and spindle rod 1 is also conceivable. This means that the spindle nut 2 is rotated instead of the spindle rod 1 being driven to rotation. In this case, the spindle rod 1 must be designed with the twist-proof locking, wherein the approaches discussed previously may be used for the twist-proof locking.

### LIST OF REFERENCE NUMERALS

D Metering device
S Spindle unit
M Drive unit
A Reservoir
K Plunger
Q Cross-sectional area of reservoir
Dᵢ Equivalent diameter of reservoir
p Spindle pitch
i Gear reduction ratio
Ci Insulin concentration
1 Spindle rod
2 Spindle nut
3 Inside wall
4 Sealing
5 O-ring
6 Outside wall
7 Locking cam
8 Housing
9 Twist-proof lock
10 Wing
11 Inside wall
12 Pull-up rod
13 Connecting needle
14 Bayonet connection
15 Motor
16 Gear
17 Planetary gear
18 Deflecting gear
19 Driving rod
20 Elongated hole
21 Stop for support on inside wall
22 Stop for support on driving rod
23 Force sensor
24 Stop disk
25 External thread
26 Internal thread
27 Spindle drive
28 Stop for support on stop disk
29 Inside radius
30 Outside radius

1. A metering device (D) for dispensing medication fluid, said metering device (D) comprising:
a) a spindle unit (S) having a constant spindle pitch (p);
b) a drive unit (M) for rotational driving of the spindle unit (S);
c) a reservoir (A) for the medication fluid, wherein the reservoir (A) has a wall (3), which defines a cross-sectional area (Q) of the reservoir (A); and
d) a plunger (K) situated in the reservoir (A), wherein the rotational driving of the spindle unit (S) causes a translational movement of the plunger (K) so that the plunger (K) is movable relative to the wall (3) of the reservoir (A) for displacement of the medication fluid,
characterized in that the product of the cross-sectional area (Q) of the reservoir (A) in units of mm² and the spindle pitch (p) of the spindle unit (S) in units of mm/angular degree is less than 0.13 mm³/angular degree, and the medication fluid is a liquid insulin in a concentration in a range of U20 to U100.

2. The metering device according to claim 1, characterized in that the medication fluid is a liquid U100 insulin and/or the volume of the reservoir (A) is in the range from 200 to 1000 mm³.

3. The metering device according to claim 1 or 2, characterized in that the cross-sectional area (Q) of the reservoir (A) is greater than 24 mm².

4. The metering device according to any of claims 1 to 3, characterized in that the product of the cross-sectional area (Q) of the reservoir (A) in units of mm² and the spindle pitch (p) of the spindle unit (S) in units of mm/angular degree is less than 0.08 mm³/angular degree, and the cross-sectional area (Q) is greater than 32.2 mm².

5. The metering device according to any of claims 1 to 4, characterized in that the spindle pitch (p) is in a range from 0.2 mm/revolution to 1.0 mm/revolution, units converted to a range of 0.00056 mm/angular degree to 0.0028 mm/angular degree.

6. The metering device according to any of claims 1 to 5, characterized in that the drive unit (M) has a motor (15) as the drive element and has a gear (16) driven by the motor (15) with a gear reduction ratio (i) for stepping down the motor angle.

7. The metering device according to claim 6, characterized in that the gear reduction ratio (i) of the gear (16) is in the range of 200 to 2000.

8. The metering device according to any of claims 1 to 7, characterized in that the spindle unit (S) has a spindle nut (2) and a spindle rod (1).

9. The metering device according to claim 8, characterized in that the spindle unit (S) is arranged in the plunger (K) and is drive by an driving rod (19) arranged on the transmission output, wherein the driving rod (19) has an axial stop (22) for supporting the spindle unit (S) in the delivered state.

10. The metering device according to claim 9, characterized in that the spindle rod (1) is fixedly connected to the plunger (K), and the spindle rod (1) is driven by the driving rod (19), wherein an external thread (25) on the spindle rod (1) engages with an internal thread (26) on a twist-proof-locking spindle nut (2), thereby forming a spindle drive (27).

11. The metering device according to claim 10, characterized in that the spindle rod (1) is retracted in a starting state prior to filling the reservoir (A), and the plunger (K) together with the spindle unit (S) can be displaced for filling the reservoir (A) with a pull-up rod (12).

12. The metering device according to claim 10, characterized in that the spindle rod (1) is extracted in a starting state prior to the filling of the reservoir (A), and the wall (3) has an axial stop (21) for the spindle nut (2), preventing any displacement of the spindle nut (2) in the direction of conveyance.

13. The metering device according to claim 12, characterized in that in a reverse rotation of the driving rod (19), the spindle nut (2) is supported on its stop (21) formed on the wall (3).

14. The metering device according to claim 9, characterized in that the spindle rod (1) is driven to rotate by the driving rod (19) wherein the spindle rod (1) has an external thread (25) and engages with an internal thread (26) of a twist-proof-locking spindle nut (2), thereby forming a spindle drive (27), and the spindle nut (2) is fixedly connected to the plunger (K), so that the plunger (K) executes only a translational movement in the delivery of medication fluid.

15. The metering device according to claim 14, characterized in that in a starting state prior to filling the reservoir (A), the spindle nut (2) is in the retracted state and the plunger (K) holding the integrated spindle unit (S) is displaceable in the forward direction and in the opposite direction by means of a pull-up rod (12) for filling the reservoir (A).

16. The metering device according to claim 14, characterized in that the twist-proof-locking spindle nut (2) is extracted in a starting state prior to filling the reservoir (A), and the wall (3) has an axial stop (21) for the spindle rod (1), preventing a displacement of the spindle rod (1) in the direction of delivery.

17. The metering device according to claim 16, characterized in that with a reverse rotation of the driving rod (19), the spindle rod (1) is supported via its stop (21) formed on the wall (3).

18. The metering device according to claim 16, characterized in that the spindle rod (1) is designed in two parts, wherein the one part is designed as a spindle rod (1) driven by the driving rod (19) and having an external thread (25), while the second part is designed as a disk (24), wherein an axial force of the spindle rod (1) is accommodated on another radius (29) in operation in reverse, and the disk (24) conducts the axial force on an outer radius (30) to the wall (3) of the reservoir (A).

19. The metering device according to any of claims 1 to 18, characterized in that the wall (3) of the reservoir (A) is cylindrical and surrounded by an outside wall (6) in the axial direction, wherein the reservoir (A) can be fixedly connected to a housing (8) by means of the outside wall (6).

20. The metering device according to claim 19, characterized in that the outside wall (6) of the reservoir (A) can be connected to the housing (8) by means of a bayonet connection (14) and the reservoir (A) in the housing (8) is thereby secured axially, wherein the bayonet connection (14) secures the reservoir (A) axially in the direction of forward movement as well as in the opposite direction.

21. The metering device according to any of claims 10 to 20, characterized in that attempts to provide a twist-proof locking of the spindle nut (2) are accomplished by means of longitudinal grooves.

22. The metering device according to any of claims 10 to 20, characterized in that the spindle nut (2) has radial wings (10) by means of which the spindle nut (2) is supported on an inside wall (11) of the outside wall (6), and the spindle nut (2) is locked in a twist-proof manner.

23. The metering device according to any of claims 1 to 22, characterized in that the metering device (D) is suitable for metering insulin in an insulin pump.

24. Use of a metering device according to any one of the preceding claims as an insulin pump, in particular for children and young people.
